(19) United States
(12) Patent Application Publication
(10) Pub. No.: US 2024/0316550 A1
(43) Pub. Date: Sep. 26, 2024

(54) METHOD AND SYSTEM FOR CHARACTERIZING AN ACOUSTIC-BASED PARTICLE MANIPULATION DEVICE

(71) Applicant: LUMICKS CA Holding B.V., Amsterdam (NL)

(72) Inventors: Douwe Kamsma, Amsterdam (NL); Felix Oswald, Amstelveen (NL); Amanda Caroline Franklin, Bristol (GB)

(21) Appl. No.: 18/574,549

(22) PCT Filed: Jun. 30, 2022
(86) PCT No.: PCT/EP2022/068076

§ 371 Date: Dec. 27, 2023

(30) Foreign Application Priority Data
Jun. 30, 2021 (NL) 2028593

(51) Int. Cl. B01L 3/00; G01H 1/00

(52) U.S. Cl. CPC B01L 3/50273; G01H 1/00; B01L 2200/0647; B01L 2300/0654; B01L 2400/0436

(57) ABSTRACT

A method is disclosed for characterizing an acoustic-based particle manipulation device comprising a sample holder having a holding space containing a fluid medium. The holding space comprises a wall surface portion. The method comprises providing a driving signal to the sample holder for generating an acoustic wave in the holding space that drives a particle at the wall surface portion, away from the wall surface portion. The method further comprises measuring vibrations of at least part of a surface of the sample holder caused by said driving signal. The method also comprises, based on the measured vibrations of said at least part of the surface, determining, for each position out of a plurality of positions at the wall surface portion and in the holding space, a value for an acoustic force that is caused by said driving signal and that acts in a direction away from the wall surface portion.

### CROSS-REFERENCE TO RELATED APPLICATION(S)

This Application is a Section 371 National Stage Application of International Application No. PCT/EP2022/068076, filed Jun. 30, 2022, and published as WO 2023/275257 A1 on Jan. 5, 2023, and further claims priority to Netherlands Patent Application No. 2028593, filed Jun. 30, 2021, the contents of which are hereby incorporated by reference in their entirety.

### FIELD OF THE INVENTION

This disclosure relates to a method and system for characterizing an acoustic-based particle manipulation device. In particular to such method and system wherein vibrations are measured of a surface of a sample holder of the acoustic-based particle manipulation device.

### BACKGROUND

Microscopic study and/or manipulation of small particles is an active field of research, in particular in case the small particles are biological specimens such as cells, organelles, few-cell bodies and the like. For biological specimens, biological processes are researched. Biological cells have an outer membrane. This membrane is the interface between the cell and its environment. The membrane therefore builds the platform for a variety of processes that involve membrane embedded biomolecules making physical contact with binding partners in the extracellular space.

Therefore, a number of techniques have been developed for studying cellular and subcellular processes by interaction with the cellular membrane, and techniques have been devised to quantify the components of the cell surface to obtain information on the specific cell type (for example to determine the type of breast cancer).

A particularly suitable technique for manipulating and/or studying small particles and in particular cellular bodies is employing of acoustic forces. It is noted that the use of acoustic forces to manipulate micron-sized particles and cells is known in general. E.g. WO 2014/200341 provides an example of an acoustic wave system for use in studying biomolecules attached to microbeads; WO 2018/083193 discloses a method, system and sample holder for manipulating and/or investigating cellular bodies; and WO 2019/212349 discloses a method for probing mechanical properties of cellular bodies. Further, G. Thalhammer et al. “Acoustic force mapping in a hybrid acoustic optical micromanipulation device supporting high resolution optical imaging”, Lab Chip 16:1523 (2016) is noted, and a summary of current research in acoustofluidics can be found in V. Marx, “Biophysics: using sound to move cells”, Nature Methods, 12(1):41 (2015). Reviews are also presented in H. Mulvana et al., “Ultrasound assisted particle and cell manipulation on-chip”, Adv. Drug Del. Rev. 65(11-12):1600 (2013); and M. Evander and J. Nilsson. “Acoustofluidics 20: Applications in acoustic trapping”, Lab on a Chip, 12:4667 (2012).

A disadvantage of acoustic-based particle manipulation devices is that their exact acoustic modes and associated pressure fields are difficult to predict. Therefore, each acoustic-based particle manipulation device is typically characterized after fabrication. As explained in Cacace et al, Digital holography as 3D tracking tool for assessing acoustophoretic particle manipulation, Optics Express, Vol. 25, No. 15, 24 Jul. 2017, (hereinafter referred to as “Cacace”) characterization of acoustic-based particle manipulation devices typically relies on particle tracking. Also see references-cited in Cacace. Unfortunately, such calibration is cumbersome as the particles need to be inserted into the acoustic-based particle manipulation device for such calibration, and withdrawn from the acoustic-based particle manipulation device thereafter.

Hence, in light of the above, there is a need in the art for improved methods for characterizing acoustic-based particle manipulation device.

### SUMMARY

To that end, a method is disclosed for characterizing an acoustic-based particle manipulation device. The acoustic-based particle manipulation device comprises a sample holder comprising a holding space containing a fluid medium. The holding space comprises a wall surface portion. The method comprises providing a driving signal to the sample holder for generating an acoustic wave in the fluid-medium-containing holding space that is suitable for driving away a particle that sits at the wall surface portion away from the wall surface portion. The method further comprises measuring vibrations of at least part of a surface of the sample holder caused by said driving signal. Preferably, vibrations of at least part of said wall surface portion are measured. The method also comprises, based on the measured vibrations of said at least part of the surface, determining, for each position out of a plurality of positions at the wall surface portion and in the holding space, a value of an acoustic force that is caused by said driving signal and that acts in a direction away from the wall surface portion, preferably the direction being substantially orthogonal to the wall surface portion.

This method advantageously does not require any particle tracking. The inventors have realized that the vibration of a surface, such as the wall surface portion, of the sample holder of an acoustic-based particle manipulation device correlates with the acoustic radiation force distribution in the holding space. The acoustic force that a particle in the holding space would experience as a result of the acoustic wave depends, amongst other things, on the properties of the particle, such as its dimensions and its compressibility. Hence, in principle, one can only determine an acoustic force for a predefined body. In any case, by simply measuring the vibrations of said surface, it is possible to characterize the acoustic-based particle manipulation device in the sense that for a given particle sitting at a given position at the wall surface portion, it can be accurately determined how large the acoustic force is that will drive the particle away from the wall surface portion when the driving signal is provided to the sample holder. This is very useful. Despite the fact that an acoustic based particle manipulation device that is configured to drive particles in an axial direction, e.g. the z-direction in FIGS. 1, 2 and 2A, is expected, based on its symmetry, to cause the same acoustic force in the axial direction for any lateral position at the wall surface portion, e.g. for any position on an xy-plane at the wall surface portion, e.g. on an xy-plane that sits slightly above the wall surface portion, this is typically not the case. First of all, the finite size of the device in the lateral dimensions causes edge effects and the purely axial piston mode may couple to bending modes which introduce lateral variations in the force as seen for example in FIG. 8 which shows that crossing the chip in the y-direction (from bottom to top in the figure) one encounters two ridges of maximum around the center of the channel. Similarly, crossing the chip in the x-direction (from left to right in the figure) one finds that the force increases to a maximum and then decreases again. Small variations in e.g. piezo alignment (shift, rotation or tilt) or small variations in electrode or contact placement etc. can cause significant shifts and alterations to this pattern of distribution of the acoustic force over the chip. Hence, during an experiment, test particles sitting at some region at the wall surface portion may very well experience a different acoustic force than identical test particles sitting at another region at the wall surface portion. An accurate characterization enables to accurately determine at which acoustic force the particle separates from the wall surface portion and thus enables to accurately determine how strong the particle is adhered to the wall surface portion.

It should be appreciated that the determined values for the acoustic force need not be absolute values. These values may be relative values in the sense that they indicate at which positions at the wall surface portion particles experience the highest acoustic forces, for example. Knowledge of the value of the acoustic force at various positions at the wall surface portion can be used to deliberately position particles at selected positions so that these particles will experience a desired acoustic force.

The method obviates the need to insert particles into an acoustic-based particle manipulation device, perform complicated particle tracking measurements, and then withdraw the particles again from the device. The method thus greatly simplifies and speeds up the characterization of acoustic-based particle manipulation devices which opens up the possibility to characterize numerous acoustic-based particle manipulation devices in only limited time. This enables large volume production of many such acoustic-based particle manipulation devices without excessive calibration effort. The method can for example be implemented in an automated production line for producing acoustic-based particle manipulation devices. The method can also ensure that, in use, acoustic-based particle manipulation devices can be operated in a way that ensures quantitative reproducibility of the forces applied during manipulation with different devices. E.g. control parameters for the driving signal may be adjusted, in use, based on the previous characterization of the device.

For devices designed to apply a force away from the wall surface on many particles in parallel where the design is such that the force is largely orthogonal to the wall surface there may be other forces acting in non-orthogonal directions, e.g. there may be lateral nodes besides the primary axial nodal plane. It may be difficult to accurately predict these lateral forces based on the vibration measurements of the surface and in general it may be difficult to accurately predict forces at a large distance from the wall surface because small variations in material properties and/or device geometry may have a large effect on the precise evolution of the acoustic field away from the wall surface. However, for devices such as these that are primarily designed to apply orthogonal forces on particles close to the wall surface, measurement of vibrations of the wall surface may be used to accurately predict the force close to the wall surface.

An average over the determined force field in at least part of the sample holder may be determined. The thus determined average force may be used as a parameter for determining quality of a sample holder and/or may be used to scale one or more properties, e.g. amplitude and/or frequency of the driving signal in order to account for differences between sample holders e.g. to ensure that similar, well determined, forces are applied to particles in different sample holders.

Further, the method can be easily performed and can therefore be performed “on-site”. The acoustic-based particle manipulation device can be characterized just before it is used in an experiment (or even after it has been used in an experiment) instead of right after fabrication, which benefits accuracy. This is especially advantageous if releasable devices are used. Such devices may only have defined resonance properties after sealing on-site because resonance properties may change slightly each time the device is opened and re-sealed.

The acoustic force of which the values are determined may be an acoustic force that would be experienced by a reference particle. Such reference particle need not be actually present in the holding space at any time. Such reference particle need not be present when the vibrations of the surface are measured, for example. For each position at the wall surface portion, the determined value of acoustic force may thus indicate a magnitude of the acoustic force that would act on such reference particle if such reference particle would sit at the position in question. Each determined value of acoustic force may in particular indicate a magnitude of a component of the acoustic force, which component is directed away from the wall surface portion, e.g. directed substantially orthogonally away from the wall surface portion. The determined values may be relative values, e.g. in the sense that they indicate that the acoustic force for some positions at the wall surface portion is higher than the acoustic force for other positions at the wall surface portion.

Even further, the acoustic force of which the values are determined may be an acoustic force that would be experienced by a reference particle if the holding space would contain some reference fluid. For each position at the wall surface portion, the determined value of acoustic force may thus indicate a magnitude of the acoustic force that would act on such reference particle if the holding space would contain the reference fluid and if such reference particle would sit at the position in question.

The reference particle is for example a silica bead with a ca 10.1 micrometer diameter (standard deviation 0.1 micrometer). The reference fluid is for example a sample fluid consisting of 1× PBS (Phosphate-buffered saline) solution supplemented with 0.02% w/v Pluronic f127+0.02% w/v Casein solution at a sample temperature in a range of 25-37 degrees Celsius.

An acoustic force determined for a reference particle and for some position at the wall surface portion may be used for determining an acoustic force that another particle would experience sitting at that position at the wall surface portion. This other particle may be a particle that is actually provided at another time in the sample holder. This may in particular apply if the properties of the reference particle are known and differ from properties of the other particle. This can be done, for example, by calculating the local (one-, two- or three dimensional) acoustic pressure gradient and/or acoustic energy density based upon the determined acoustic force on the reference particle. Conversion from acoustic force to acoustic energy density and back can for example be done using the following equation taken from Bruus, H. (2012) ‘Acoustofluidics 7: The acoustic radiation force on small particles’, Lab on a Chip, The Royal Society of Chemistry, pp. 1014-1021. doi: 10.1039/c2lc21068a:

$${F}_{z}^{r a d} = - {\partial }_{z} {U}^{r a d} = 4 π Φ ( κ , ρ ) k {a}^{3} {E}_{a c} sin ( 2 k z )$$

here F_{z}^{rad} is the acoustic radiation force, U^{rad} is the acoustic radiation force potential, Φ is the acoustic contrast factor, κ is the compressibility ratio, ρ is the density ratio, k is the wavenumber, a is the particle radius, E_{ac} is the acoustic energy density, and z is the distance from the wall.

In light of the above, it should be appreciated that determining a value of an acoustic force acting on a (virtual) reference particle may be embodied as determining a value of a direction derivative of acoustic pressure along the direction away from the wall surface portion.

It should be noted that any acoustic pressure gradient or acoustic force referred to herein, may have a direction in 3D space in which the acoustic field amplitude changes most rapidly. Since both acoustic pressure and acoustic velocity determine the acoustic force acting on a particle one can also speak of the directional derivative of complex acoustic pressure.

The acoustic-based particle manipulation device may comprise an oscillator, which may comprise a transducer for converting an electrical driving signal, such as an oscillating voltage, into mechanical vibrations in order to generate an acoustic signal in the holding space. Preferably, the generated acoustic wave is a bulk acoustic standing wave between two opposite surfaces defining the holding space (see e.g. FIG. 2A). Also, preferably, the acoustic wave in the holding space is suitable for driving away a particle from the wall surface portion in a direction that is substantially orthogonal to the wall surface portion. In an example an optimal force generation for particular studies may be achieved by selecting acoustic cavity parameters and the frequency/wavelength of the acoustic wave in order to create a maximum pressure gradient at the functionalised wall surface, e.g. by ensuring that the distance from the wall surface to the acoustic node is approximately ¼ wavelength.

The acoustic wave may be an ultrasonic acoustic wave, e.g. having a frequency in a range of 1-30 MHz, preferably in a range 5-20 MHz. The measured vibrations of the surface are typically vibrations in the direction away from the wall surface portion.

Preferably, the method also comprises storing on a computer-readable storage medium the determined values for the acoustic force in association with their associated respective positions, optionally in association with an identifier of the acoustic-based particle manipulation device. The directly obtained product of this method embodiment is thus a computer-readable storage medium having stored thereon the values for the acoustic force in association with their associated respective positions. The acoustic-based particle manipulation device itself may comprise such computer-readable storage medium. Additionally or alternatively, the computer-readable storage medium sits remote from the acoustic-based particle manipulation device, e.g. at a remote server system.

In an embodiment, measuring the vibrations of the at least part of the surface is performed using an imaging technique. Preferably, measuring the vibrations of the at least part of the surface is performed using microscopy, preferably holographic microscopy, more preferably digital holographic microscopy.

As said, in an embodiment, measuring the vibrations of the at least part of the surface is performed using digital holographic microscopy. Digital holographic microscopy has proven an effective technology for measuring vibrations, as for example described in Telschow et al. Full-Field Imaging of Gigahertz Film Bulk Acoustic Resonator Motion, IEEE transactions on ultrasonics, ferroelectrics, and frequency control, vol. 50, no. 10, October 2003 and in Cuche et al., Simultaneous amplitude-contrast and quantitative phase-contrast microscopy by numerical reconstruction of Fresnel off-axis holograms, Applied Optics, Vol. 38, No. 34, 1 Dec. 1999 and in Cuche et al., Digital holography for quantitative phase-contrast imaging, Optics Letters, Vol. 24, No. 5, Mar. 1, 1999.

The method according to any of the preceding claims, wherein measuring the vibrations of the at least part of the surface is performed using laser doppler vibrometry and/or phase sensitive optical coherence tomography and/or optical coherence phase microscopy.

Laser doppler vibrometry is for example described in Baudoin, et. al. (2020). Spatially selective manipulation of cells with single-beam acoustical tweezers. Nature Communications, 11(1), 1-10.

Phase sensitive optical coherence tomography and optical coherence phase microscopy are for example described in Helderman, F., Haslam, B., de Boer, J. F., & de Groot, M. (2013), Three-dimensional intracellular optical coherence phase imaging. Optics Letters, 38(4), 431-433 and Joo, C., Akkin, T., Cense, B., Park, B. H., & De Boer, J. F. (2005), Spectral-domain optical coherence phase microscopy for quantitative phase-contrast imaging, Optics Letters, 30(16), 2131-2133.

In case the vibrations are measured using light that reflects back from the surface of interest, then preferably that light has a coherence length of 1-100 micrometers, preferably 5-50 micrometers, more preferably 10-20 micrometers. Such relatively short coherence is advantageous in that it enables to accurately measure vibrations of the surface, even if other surfaces nearby that surface also reflect the light. The relatively short coherence length may namely prevent, at least to some extent, that light reflected from the surface of interest and light reflected from a nearby surface interfere with each other, which may cause measurement artifacts. As referred to herein, “coherence length” may be understood as the optical path length difference, as measured using a Michelson interferometer, of a self-interfering laser beam which corresponds to

$$\frac{1}{e} = 37 \%$$

fringe visibility, where the fringe visibility is defined as

$$V = \frac{{I}_{max} - {I}_{min}}{{I}_{max} + {I}_{min}}$$

where Iₘₐₓ resp. Iₘᵢₙ is the maximum resp. minimum fringe intensity.

In an embodiment, each position out of the plurality of positions is at a distance from the wall surface portion.

During an actual particle experiment, such as a particle adhesion test, particles under investigation, such as cellular bodies, are typically not directly contacting the wall surface portion. The particles under investigation are typically adhered to other particles, such as other cells forming a functionalized layer, which, in turn, are adhered to the wall surface portion. Typically, the aim of the particle adhesion test is to assess how strong the particles under investigation adhere to these other particles. In any case, typically, the particles under investigation will sit at a distance from the wall surface portion. Hence, the positions at the wall surface portion for which the acoustic force values are determined, are preferably slightly above the wall surface portion, e.g. 2-10 micrometers above the wall surface portion. A particle sitting at the wall surface portion may be understood as the particle being adhered to the wall surface portion, optionally via one or more other particles, e.g. another cell, that is or are adhered to the wall surface portion.

In an embodiment, measuring vibrations of the at least part of the surface comprises determining, for each point of a plurality of points on the surface, an amplitude value indicative of amplitude with which the point in question vibrates.

The inventors have realized that, especially for measurements of adhesion forces close to the surface of an acoustic manipulation device, a measurement of the vibration amplitude of the surface (i.e. the amplitude of a plurality of points on the surface) yields a good quantitative prediction for the acoustic radiation force experienced by a test particle at, e.g. directly above, these points on the surface. E.g. the force can be estimated to be proportional to the square of the amplitude of the surface displacement. The proportionality constant between the square amplitude of the surface displacement and the force applied to a test particle depends on the acoustic contrast factor and the size of the test particle. The force on a different particle can be calculated as long as the properties of the particle are known. Therefore, the method allows every acoustic manipulation device to be calibrated such that experiments and measured forces can be compared between different devices

A point on a surface as used in this disclosure may be indicative of an area on the surface. The amplitude with which the point in question vibrates may be an amplitude in a direction away from the wall surface portion, e.g. in a direction orthogonal to the wall surface portion.

This embodiment may comprise determining the value for acoustic force at the respective positions based on amplitude measurements of respective points on the surface. To illustrate, for each position out of the plurality of positions, determining its acoustic force value may be performed based on one or more amplitude measurements of one or more respective points on the surface. Thus, measured vibration of several points on the surface may be used for determining the acoustic force value at one position at the wall surface portion. This allows to take into account complex behavior of the acoustic wave, such as smoothing effect due to acoustic field propagation.

The method according to any of the preceding claims, wherein said surface is a surface in the holding space and/or is a surface at least partially defining the holding space and/or is a surface adjacent the holding space and/or is the wall surface portion.

Preferably, the surface is relatively close to the holding space so that the acoustic pressure in the holding space can be accurately determined.

In an embodiment, the step of determining, for each position, a value for acoustic force is performed based on reference data associating reference amplitude values with respective reference values of acoustic force.

Each reference amplitude value may be understood to be indicative of an amplitude with which a reference point on a reference surface of a reference sample holder of a reference acoustic-based particle manipulation device vibrates. Each reference value of acoustic force may be indicative of acoustic force in a direction away from a reference wall surface portion at a reference position in a reference holding space of the reference sample holder.

The reference data thus link measured amplitude values at respective points on the surface to respective values of acoustic force in a direction away from the wall surface portion.

Further, each reference amplitude-reference acoustic force value pair may be associated with one or more parameters, such as current and/or voltage and/or power and/or frequency, of a reference driving signal that was used for measuring the pair. This allows to determine for a given position in the holding space of a to-be-characterized particle manipulation device and for a given driving signal provided to the sample holder an acoustic force value on the basis of vibration measurements.

In an embodiment, the method comprises obtaining said reference data. In an embodiment, this step comprises
providing a reference acoustic-based particle manipulation device comprising a reference sample holder comprising a reference holding space containing a reference fluid medium, the reference holding space comprising a reference wall surface portion, and
providing a reference driving signal to the reference sample holder for generating a reference acoustic field in the reference holding space that is suitable for driving a particle that sits at the reference wall surface portion, away from the reference wall surface portion, and
measuring vibrations of at least part of a reference surface of the reference sample holder caused by said reference driving signal, and
measuring, for each position out of a plurality of positions at the reference wall surface portion and in the reference holding space, a reference value of an acoustic force that is caused by said reference driving signal and that acts in a direction away from the reference wall surface portion, preferably the direction being substantially orthogonal to the reference wall surface portion.

In principle, the reference data can be obtained by performing such reference measurement on only one reference acoustic/based particle manipulation device. The thus obtained reference data can then be used for interpreting vibration measurements of any other acoustic-based particle manipulation device.

This embodiment may comprise storing the measured reference acoustic force values in association with the measured vibrations of the at least part of the reference surface.

In this embodiment, preferably, measuring the reference values of the acoustic force in a direction away from the reference wall surface portion is performed using particle tracking techniques known in the art, which involves tracking how particles move through the reference holding space and fitting the bead trajectories to physical models in order to determine the forces as experienced by the particles.

Measuring vibrations of the reference surface may comprise determining, for each point of a plurality of points on the reference surface, a reference amplitude value indicative of amplitude with which the point in question vibrates, preferably in the direction away from the reference wall surface portion.

In an embodiment, the method comprises
providing a reference acoustic-based particle manipulation device comprising a reference sample holder comprising a reference holding space containing a reference fluid medium, the reference holding space comprising a reference wall surface portion, and
providing a first reference driving signal to the reference sample holder for generating a first reference acoustic wave in the reference holding space that is suitable for driving a particle that sits at the reference wall surface portion, away from the reference wall surface portion, and
measuring first vibrations of at least part of a reference surface of the reference sample holder caused by said first reference driving signal, and
measuring, for each position out of a plurality of positions at the reference wall surface portion and in the reference holding space, a first reference value for the acoustic force that is caused by said first reference driving signal and that acts in a direction away from the reference wall surface portion, preferably the direction being substantially orthogonal to the wall surface portion, and
providing a second reference driving signal to the reference sample holder for generating a second reference acoustic wave in the reference holding space that is suitable for driving a particle that sits at the reference wall surface portion, away from the reference wall surface portion, and
measuring second vibrations of at least part of a reference surface of the reference sample holder caused by said second reference driving signal, and
measuring, for each position out of a plurality of positions at the reference wall surface portion and in the reference holding space, a second reference value of acoustic force that is caused by said second reference driving signal and that acts in a direction away from the reference wall surface portion, preferably the direction being substantially orthogonal to the wall surface portion.

The first and second reference driving signals are different, for example in terms of their current and/or voltage and/or frequency. Preferably, this embodiment comprises storing the determined first reference values of the directional derivate in association with their associated respective positions in the holding space and in association with one or more values of respective parameters, such as current and/or voltage and/or frequency, of the first reference driving signal; and storing the determined second reference values for the directional derivates in association with their associated respective positions in the holding space and in association with one or more values of respective parameters of the second reference driving signal. Each reference amplitude-reference acoustic force value pair may be associated with one or more parameters, such as current and/or voltage and/or power and/or frequency, of the reference driving signal that was used for measuring the pair.

In an embodiment, the method comprises storing the determined values for the acoustic force on a no-transitory computer-readable storage medium in association with respective indications of their respective associated positions in the holding space, wherein, preferably, the acoustic-based particle manipulation device comprises said computer-readable storage medium.

Preferably, the acoustic force values are also stored in association with one or more values of respective parameters of the driving signal, such as the driving signal's voltage, current, frequency, electrical power, etc. Understandably, the acoustic force values are stored in association with an identifier of the acoustic particle manipulation device which is characterized. However, this is not strictly required, for example if the particle manipulation device itself comprises the storage medium.

Optionally, after the characterization, the fluid is removed from the holding space. The directly obtained product of this method embodiment may thus be an acoustic-based particle manipulation device comprising a sample holder comprising a holding space for containing a fluid medium, wherein the acoustic-based particle manipulation device comprises a computer-readable storage medium having stored thereon for each position out of a plurality of positions at the wall surface portion and in the holding space, an associated value of acoustic force that acts in a direction away from the wall surface portion.

In an embodiment, the method comprises
providing a first driving signal to the sample holder for generating a first acoustic wave in the fluid-medium-containing holding space that is suitable for driving a particle that sits at the wall surface portion, away from the wall surface portion, and
measuring first vibrations of at least part of a surface of the sample holder caused by said first driving signal, and
based on the measured first vibrations of said at least part of the surface, determining, for each position out of a plurality of positions at the wall surface portion and in the holding space, a first value of the acoustic force that is caused by said first driving signal and that acts in a direction away from the wall surface portion, preferably the direction being substantially orthogonal to the wall surface portion, and
providing a second driving signal to the sample holder for generating a second acoustic wave in the fluid-medium-containing holding space that is suitable for driving a particle that sits at the wall surface portion, away from the wall surface portion, and
measuring second vibrations of at least part of a surface of the sample holder caused by said second driving signal, and
based on the measured second vibrations of said at least part of the surface, determining, for each position out of a plurality of positions at the wall surface portion and in the holding space, a second value for the acoustic force that is caused by said second driving signal and that acts in a direction away from the wall surface portion, preferably the direction being substantially orthogonal to the wall surface portion.

This embodiment enables to characterize the device for various driving signals. Of course, many more driving signals than two may be provided and the device may be characterized for each of them. This embodiment thus allows to characterize the device in the sense that for a given particle sitting at a given position at the wall surface portion while a given driving signal is provided to the sample holder, it can be accurately determined how large the acoustic force is that will drive the particle away from the wall surface portion.

The first and second driving signals are different, for example in terms of their current and/or voltage and/or frequency and/or waveform. Preferably, this embodiment comprises storing the determined first values of the directional derivate in association with their associated respective positions in the holding space and in association with one or more values of respective parameters, such as current and/or voltage and/or frequency, of the first driving signal; and storing the determined second values for the directional derivates in association with their associated respective positions in the holding space and in association with one or more values of respective parameters of the second driving signal.

One aspect of this disclosure relates to a system comprising an acoustic-based particle manipulation device, a signal provisioning system, a vibration measurement system and, optionally, a data processing system. The acoustic-based particle manipulation device comprises a sample holder comprising a holding space containing a fluid medium, the holding space comprising a wall surface portion. The signal provisioning system is suitable for providing a driving signal to the sample holder for generating an acoustic field in the fluid-medium-containing holding space that is suitable for driving a particle that sits at the wall surface portion, away from the wall surface portion. The vibration measurement system is suitable for measuring vibrations of at least part of a surface of the sample holder caused by said driving signal. Further, the optional data processing system is configured to determine, based on the measured vibrations of said at least part of the surface, for each position out of a plurality of positions at the wall surface portion and in the holding space, a value for the acoustic force that is caused by said driving signal and that acts in a direction away from the wall surface portion, preferably the direction being substantially orthogonal to the wall surface portion.

One aspect of this disclosure relates to a computer-implemented method for characterizing an acoustic-based particle manipulation device. The device comprises a sample holder comprising a holding space containing a fluid medium, the holding space comprising a wall surface portion. The computer-implemented method comprises obtaining vibration data indicating, for each point of a plurality of points on a surface of the sample holder, a measured amplitude value indicative of amplitude with which the point in question vibrates as a result of a driving signal provided to the sample holder. The computer-implemented method comprises, based on the vibration data, determining, for each position out of a plurality of positions at a wall surface portion of a holding space, the plurality of positions being in the holding space, a value for the acoustic force that is caused by said driving signal and that acts in a direction away from the wall surface portion, preferably the direction being substantially orthogonal to the wall surface portion.

For each point, its measured amplitude value may be indicative of amplitude with which the point in question vibrates in a direction away from the wall surface portion, e.g. in a direction substantially orthogonal to the wall surface portion.

The step of determining values for the acoustic force for a plurality of positions may be performed based on reference data associating reference amplitude values with reference values for the acoustic force as described herein. The computer-implemented method naturally comprises obtaining said reference data.

Additionally or alternatively, the step of determining values for the acoustic force for a plurality of positions may be performed based on theoretical models that allow to determine acoustic force values based on measured vibrations, e.g. based on measured amplitude values.

The computer-implemented may comprise any method step disclosed herein that can be executed by a data processing system. For example, the computer-implemented may comprise providing a driving signal to the sample holder, e.g. by controlling a transducer, and/or measuring the vibrations of the at least part of the surface, e.g. by controlling a vibration measurement system, and/or obtaining reference data and/or storing data on a computer-readable storage medium.

One aspect of this disclosure relates to a data processing system that is configured to perform any of the computer-implemented methods disclosed herein.

One aspect of this disclosure relates to a computer program comprising instructions, which, when the instructions are executed by a data processing system, cause the data processing system to perform any of the computer-implemented methods disclosed herein.

One aspect of this disclosure relates to a non-transitory computer-readable storage medium having stored thereon any of the computer programs disclosed herein.

One aspect of this disclosure relates to a computer comprising a computer readable storage medium having computer readable program code embodied therewith, and a processor, preferably a microprocessor, coupled to the computer readable storage medium, wherein responsive to executing the computer readable program code, the processor is configured to perform any of the computer-implemented methods disclosed herein.

One aspect of this disclosure relates to a computer program or suite of computer programs comprising at least one software code portion or a computer program product storing at least one software code portion, the software code portion, when run on a computer system, being configured for executing any of the computer-implemented methods disclosed herein.

One aspect of this disclosure relates to a non-transitory computer-readable storage medium storing at least one software code portion, the software code portion, when executed or processed by a computer, is configured to perform any of the computer-implemented methods disclosed herein.

One aspect of this disclosure relates to a non-transitory, computer-readable storage medium obtainable by performing any of the methods described herein that involve storing data on a computer-readable storage medium.

As will be appreciated by one skilled in the art, aspects of the present invention may be embodied as a system, a method or a computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Functions described in this disclosure may be implemented as an algorithm executed by a processor/microprocessor of a computer. Furthermore, aspects of the present invention may take the form of a computer program product embodied in one or more computer readable medium(s) having computer readable program code embodied, e.g., stored, thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples of a computer readable storage medium may include, but are not limited to, the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of the present invention, a computer readable storage medium may be any tangible medium that can contain, or store, a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber, cable, RF, etc., or any suitable combination of the foregoing. Computer program code for carrying out operations for aspects of the present invention may be written in any combination of one or more programming languages, including an object oriented programming language such as Java™, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer, or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the present invention are described below with reference to flowchart illustrations and/or block diagrams of methods, apparatus (systems), and computer program products according to embodiments of the present invention. It will be understood that each block of the flowchart illustrations and/or block diagrams, and combinations of blocks in the flowchart illustrations and/or block diagrams, can be implemented by computer program instructions. These computer program instructions may be provided to a processor, in particular a microprocessor or a central processing unit (CPU), of a general purpose computer, special purpose computer, or other programmable data processing apparatus to produce a machine, such that the instructions, which execute via the processor of the computer, other programmable data processing apparatus, or other devices create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

The flowchart and block diagrams in the figures illustrate the architecture, functionality, and operation of possible implementations of systems, methods and computer program products according to various embodiments of the present invention. In this regard, each block in the flowchart or block diagrams may represent a module, segment, or portion of code, which comprises one or more executable instructions for implementing the specified logical function(s). It should also be noted that, in some alternative implementations, the functions noted in the blocks may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustrations, and combinations of blocks in the block diagrams and/or flowchart illustrations, can be implemented by special purpose hardware-based systems that perform the specified functions or acts, or combinations of special purpose hardware and computer instructions.

Moreover, a computer program for carrying out the methods described herein, as well as a non-transitory computer readable storage-medium storing the computer program are provided. A computer program may, for example, be downloaded (updated) to the existing data processing systems or be stored upon manufacturing of these systems.

Elements and aspects discussed for or in relation with a particular embodiment may be suitably combined with elements and aspects of other embodiments, unless explicitly stated otherwise. Embodiments of the present invention will be further illustrated with reference to the attached drawings, which schematically will show embodiments according to the invention. It will be understood that the present invention is not in any way restricted to these specific embodiments.

### BRIEF DESCRIPTION OF THE DRAWINGS

Aspects of the invention will be explained in greater detail by reference to exemplary embodiments shown in the drawings, in which:

FIG. 1 is a schematic drawing of a system comprising a particle manipulation device that can be characterized using the methods disclosed herein;

FIG. 2 shows a particle manipulation device in more detail;

FIG. 2A shows the holding space of a particle manipulation device in more detail;

FIG. 3 shows a simplified schematic circuit of an electrical setup of an embodiment of a manipulation system;

FIG. 3A shows a complex phase diagram of the system of FIG. 3;

FIG. 4 illustrates a system, according to an embodiment, for characterizing a particle manipulation device, wherein the system comprises a digital holography setup;

FIG. 4A illustrates operation of the digital holography setup according to an embodiment;

FIG. 5 illustrates a system, according to an embodiment, for characterizing a particle manipulation device;

FIG. 6 illustrates a system, according to an embodiment, for characterizing a particle manipulation device, wherein the system comprises a laser Doppler vibrometer;

FIG. 7A is a flow chart illustrating a method according to an embodiment;

FIG. 7B is a flow chart illustrating a method according to an embodiment for obtaining reference data

FIGS. 8 and 9 illustrate measurements comparing methods disclosed herein with prior art methods;

FIG. 10 illustrates performed simulations of vibrations and acoustic force;

FIGS. 11A and 11B illustrate a measurement comparing different coherence lengths for the light used to measure vibrations of the surface;

FIG. 11C illustrates that a shorter coherence length of the light used to measure the vibrations may yield more accurate results;

FIG. 12 illustrates a data processing system according to an embodiment.

### DETAILED DESCRIPTION OF THE DRAWINGS

In the figures identical reference numbers indicate the same or similar elements.

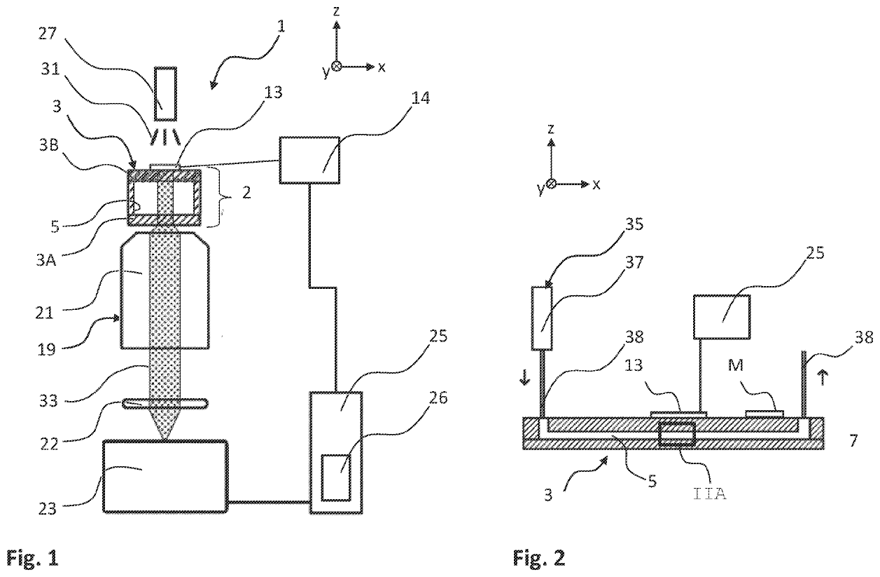

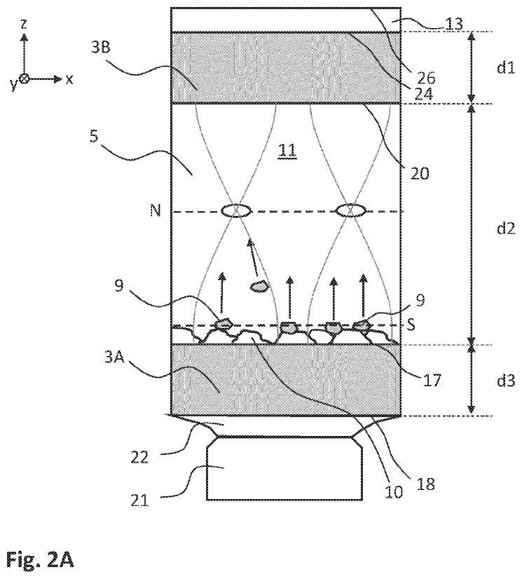

FIG. 1 is a schematic drawing of a system 1 comprising a particle manipulation device 2 that can be characterized using the methods disclosed herein. FIG. 2 is a cross section of a sample holder and FIG. 2A is a detail of the sample holder of FIG. 2 as indicated with “IIA”. How the particle manipulation device 2 can be used to test the adhesion or particles will be explained with reference to FIGS. 1, 2 and 2A.

The particle manipulation device 2 comprises a sample holder 3 comprising a holding space 5 containing a fluid medium 11. The holding space 5 comprises a wall surface portion 17. The holding space 5 is suitable for holding one or more particles of interest, such one or more biological cellular bodies 9. What is present in the holding space may be collectively referred to as the sample. It is noted that also, or alternatively, other types of particles like microspheres could be used, possibly attached to biological cellular bodies 9. The fluid 11 preferably is a liquid. In response to a driving signal as provided by a signal provisioning system to the sample holder, an acoustic wave is generated in the fluid-medium-containing holding space 5 that is suitable for driving a particle that sits at the wall surface portion 17, away from the wall surface portion 17. The signal provisioning system in the depicted embodiment comprises an acoustic wave generator 13, such as a piezo element, connected with the sample holder 2, and a system 14 comprising a data processing system and power supply (not shown).

During a particle adhesion test, the wall surface portion 17 is typically functionalized in the sense that cellular bodies are present on it and in that particles under investigation adhere to these cellular bodies. The wall surface portion 17 may also or alternatively be functionalized using other specific molecules and/or surface treatments. Typically, the particle manipulation device is used to measure the adhesion forces of particles to a specific surface. This adhesion force may for example be the cellular binding avidity in case both the functionalized layer and the particles are cells but also other interactions may be probed e.g. the surface portion may be functionalized with antibodies, biological materials such as fibronectin or collagen, atomic monolayers such as gold etc. The particles may be cells but they may also be (functionalized) particles such as polymer or glass microspheres, lipid vesicles, or any other particles with sufficient size and acoustic contrast with respect to the medium to allow acoustic manipulation of such particles. A further wall, e.g. opposite wall, may also or alternatively be functionalized in the same way as the wall surface portion 17.

The shown system 1 comprises a microscope 19 with an objective 21 and a camera 23 connected with a computer 25 comprising a controller and a memory 26. The computer 25 may also be programmed for tracking one or more of the cellular bodies based on signals from the camera 23 and/or for performing microscopy calculations and/or for performing analysis associated with (superresolution) microscopy and/or video tracking, which may be sub-pixel video tracking. The computer or another controller (not shown) may be connected with other parts of the system 1 (not shown) for controlling at least part of the microscope 19 and/or another detector (not shown). In particular, the computer 25 may be connected with one or more other parts of the system such as the acoustic wave generator 13, the power supply and/or controller 14 thereof (both as shown in FIG. 1), the light source, a temperature control, sample fluid flow control, etc. (none shown).

The system 1 further comprises a light source 27. The light source 27 may illuminate particles that sit at the wall surface portion 17 using any suitable optics (not shown) to provide a desired illumination intensity and intensity pattern, e.g. plane wave illumination, Köhler illumination, etc., known per se. Here, in the system light 31 emitted from the light source 27 is directed through the acoustic wave generator 13 to the sample holder 3 and sample light 33 from the sample is transmitted through the objective 21 and through an optional tube lens 22 and/or further optics (not shown) to the camera 23. The objective 21 and the camera 23 may be integrated. In an embodiment, two or more optical detection tools, e.g. with different magnifications and/or components related to spectral and/or polarization properties, may be used simultaneously for detection of sample light 33, e.g. using a filter and/or a beam splitter.

In another embodiment, not shown but discussed in detail in WO 2014/200341, the system comprises a partially reflective reflector and light emitted from the light source is directed via the reflector through the objective and through the sample, and light from the sample is reflected back into the objective, passing through the partially reflective reflector and directed into a camera via optional intervening optics. Further embodiments are apparent to the reader.

The sample light 33 may comprise light 31 affected by the particles under investigation (e.g. scattered and/or absorbed) and/or light emitted by one or more portions of the sample itself e.g. by chromophores and/or fluorophores attached to the cellular bodies 9.

Some optical elements in the system 1 may be at least one of partly reflective, dichroic (having a wavelength specific reflectivity, e.g. having a high reflectivity for one wavelength and high transmissivity for another wavelength), polarisation selective and otherwise suitable for the shown setup. Further optical elements e.g. lenses, prisms, polarizers, diaphragms, reflectors etc. may be provided, e.g. to configure the system 1 for specific types of microscopy.

The sample holder 3 may be formed by a single piece of material with a channel inside, e.g. glass, injection moulded polymer, etc. (not shown) or by fixing different layers of suitable materials together more or less permanently, e.g. by welding, glass bond, gluing, taping, clamping, etc., such that a holding space 5 is formed in which the fluid 11 contains one or more particles under investigation, at least during the duration of an experiment. As shown in FIGS. 1 and 2, the sample holder 3 may comprise a part 3A that has a recess being, at least locally, U-shaped in cross section and a cover part 3B to cover and close (the recess in) the U-shaped part providing an enclosed holding space 5 in cross section. A monolithic sample holder, at least at the location of the acoustic wave generator 13, may be preferred over an assembled sample holder for improving acoustic coupling, reducing losses and/or preventing local variations.

As shown in FIG. 2, the sample holder 3 is connected to an optional fluid flow system 35 for introducing fluid into the holding space 5 of the sample holder 3 and/or removing fluid from the holding space 5, e.g. for flowing fluid through the holding space (see arrows in FIG. 2). The fluid flow system 35 may comprise a manipulation and/or control system, possibly associated with the computer 25. The fluid flow system 35 may comprise one or more of reservoirs 37, pumps, valves, and conduits 38 for introducing and/or removing one or more fluids, sequentially and/or simultaneously. The sample holder 3 and the fluid flow system 35 may comprise connectors, which may be arranged on any suitable location on the sample holder 3, for coupling/decoupling without damaging at least one of the parts 3, 35, and preferable for repeated coupling/decoupling such that one or both parts 3, 35 may be reusable thereafter. Further, an optional machine-readable mark M or other identifier is attached to the sample holder 3. Further, a computer-readable storage may be attached onto the sample holder (not shown). Such sample holder may have stored information obtained during the calibration methods described herein, such as the determined values of the acoustic force on said computer-readable storage medium in association with their position in the holding space. Each value of the acoustic force may be expressed as, and therefore also stored in the form of, a value for directional derivative of acoustic pressure along a direction away from the wall surface portion.

FIG. 2A is a schematic of a number of particles 9, such as cellular bodies, in the sample holder 3 of FIG. 2. At least part of the wall surface portion 17 of the sample holder 3 is functionalized in the sense that e.g. it is covered with biological cells 10 preferably of a different type to which the particles of interest 9 may adhere. Also shown is part of the microscope lens 21 and an optional immersion fluid layer 22 for improving image quality.

On providing an, optionally periodic, driving signal to the sample holder, e.g. by providing a control signal to acoustic wave generator 13, an acoustic wave, e.g. an acoustic standing wave, is generated in the holding space 5. The signal may be selected, as indicated, such that an antinode of the wave is generated at or close to the wall surface 17 (of the sample holder 3) and a node N of the wave W away from the wall surface 17, generating a local maximum force F on the particles 9 at and/or near the wall surface 17 towards the node. Thus, application of the driving signal may serve to probe adhesion of the particles 9 to the surface 17 and/or to any functionalised layer on it in dependence of the force. The driving signal can namely cause the particles 9 that are present at the wall surface portion 17 and optionally adhered to a functionalized layer on the wall surface portion, to experience an acoustic force of certain magnitude that drives the particles away from the wall surface portion, namely towards one of the nodes N. Based on, for example, the images as obtained by camera 23, it can be determined when particles detach from such functionalized layer on the wall surface 17. The moment of detachment of a particle can be linked to the acoustic force that the particle experienced at that moment. During an experiment it is, of course, accurately monitored which driving signal is applied to the sample holder at which time and/or which acoustic force the particles experience at which time. In this way, the adhesion of particles can be tested. It will be understood that the acoustic forces that the particles experience at the wall surface portion 17, e.g. when they are adhered to a functionalized layer on the wall surface portion, should be known very accurately. Unfortunately, due to, amongst other things, uncontrollable variations in material properties and manufacturing tolerances, the force is not constant at the wall surface portion 17, i.e. not constant across a plane parallel to and at the wall surface portion 17. More importantly, the generated force field differs from particle manipulation device to particle manipulation device. Hence, each acoustic-based particle manipulation device should be accurately characterized before use. Fortunately, the generated force field is relatively stable over time so that characterization (long) before an actual experiment is indeed possible.

Preferably, for each of a plurality of positions at the wall surface portion 17, the value for the acoustic force acting in a direction away from the wall surface is known. These positions may be positions on the wall surface portion. However, typically, these positions are at some distance from the wall surface portion because the particles under investigation may be attached to a functionalized layer 10 on the wall surface portion 17. In FIG. 2A, the particles of interest 9 lie substantially in the plane S parallel to the indicated xy-plane. Hence, preferably, the characterization of this particle manipulation device provides information at which positions on the S plane, the acoustic force in a direction away from the wall surface portion 17, e.g. in the indicated z-direction, has which values. Preferably, the plurality of points span the entire plane S and are positioned close to each other such that a high-resolution characterization of the particle manipulation device can be achieved, e.g. in the sense that even for closely positioned points on plane S different values for the acoustic force can be resolved.

Preferably, vibrations of a surface are measured that is near the holding space 5, such as wall surface portion 17. However, it is also possible to measure vibrations of for example the surface 18 between an optional immersion fluid layer 22 and sample holder and/or the surface formed by a functionalized layer 10 on the wall surface portion 17 in the holding space 5 and/or the surface 20 between holding space and cover part 3B and/or the surface 24 between the cover part 3B and transducer 13 and/or the surface 26 of transducer 13. In general, measuring vibrations of a surface may comprise determining, for each point of a plurality of points on such surface, an amplitude value indicative of amplitude with which the point in question vibrates. The vibration measurements referred to herein are preferably performed with high resolution. In principle, the more points are measured, the higher the resolution of the characterization that can be achieved and the higher the fidelity of the characterization will be.

In an example an optimal force generation for particular studies may be achieved by selecting acoustic cavity parameters and the frequency/wavelength of the acoustic wave in order to create a maximum pressure gradient at the wall surface portion 17, e.g. by ensuring that the distance from the wall surface to the acoustic node is approximately ¼ wavelength.

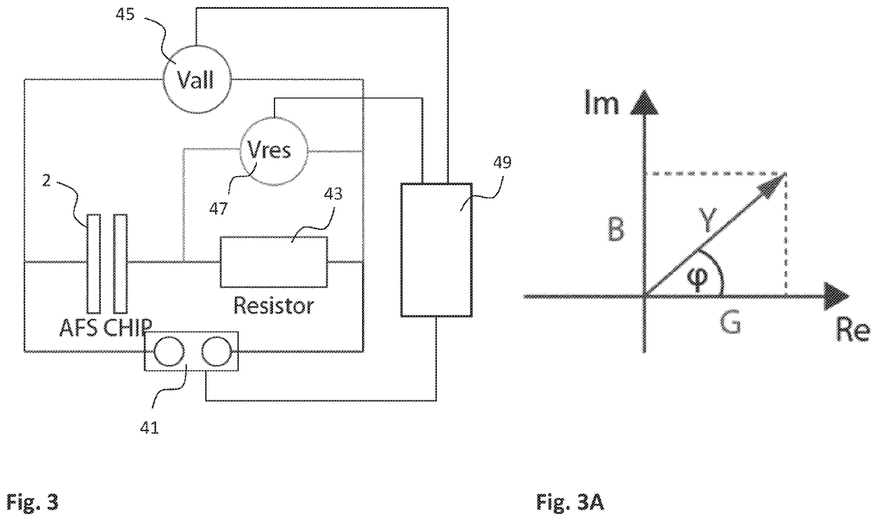

FIG. 3 shows a simplified schematic circuit of an electrical setup of the system 1. Here, the particle manipulation device 2 including sample holder 3 and acoustic wave generator 13 are considered together as a functional unit AFS Chip indicated at 2, having an impedance Z. A power supply 41 is provided for generating a periodic driving signal, having a signal frequency, a signal amplitude and a signal power. The power supply 41 is connected to the particle manipulation device 2, which may be referred to as a chip, in series with a reference resistor 43. Voltage measurement devices 45, 47 for measuring Vall and Vres are provided as indicated. The power supply 41 and voltage measurement devices 45, 47 are connected to a controller 49, which may comprise an analog-to-digital converter (ADC) and/or which may be connected with, or be part of, the controller 14 and/or the computer 25. Vchip, the voltage across the acoustic wave generator, can then also be calculated as Vchip=Vall−Vres.

When providing an oscillating driving voltage Vin by the power supply 41 to the particle manipulation device 2, a phase difference φ between Vall and Vres will occur, which may be measurable. The following values may be determined (see also the complex phase diagram in FIG. 3A):

$$Impedance : \vert Z \vert = ( Vchip ) Rres / Vres$$

$$Admittance : \vert Y \vert = 1 / \vert Z \vert$$

$$wherein Complex admittance : Y = \vert Y \vert exp ( - j φ ) = G + jB$$

$$Susceptance : B = \vert Y \vert sin ( - φ )$$

$$Conductance : G = \vert Y \vert cos ( φ )$$

The particle manipulation device 2 has certain resonance frequencies. At each resonance frequency, the conductance is at a maximum.

The acoustic-based particle manipulation devices described herein may also be referred to a as acoustic-based particle adhesion test devices and may be understood to be acoustic and/or microfluidic chips. As explained these devices can be used to apply a force to particles that are present on an, optionally functionalized, wall surface portion. This allows for interesting experiments. For example, by applying forces to immune cells bound to a layer of tumor cells on the wall surface portion and by simultaneously imaging the cells and determining unbinding events one can characterize the binding force of the immune cells on the tumor cells. This binding force, or binding avidity, is an essential parameter in the process of immune recognition. In another example molecules, such as for example DNA molecules, may be bound to the wall surface portion and beads, e.g. 10 um polystyrene beads, may be attached to the other end of the DNA molecules. Acoustic forces may be used to push the beads away from the wall surface portion and stretch the DNA molecules. By measuring the height of the beads above the surface, e.g. by using video microscopy, one may determine mechanical signatures of the molecules and/or changes in these mechanical signatures induced by e.g. other molecules such as proteins that bind to the molecules.

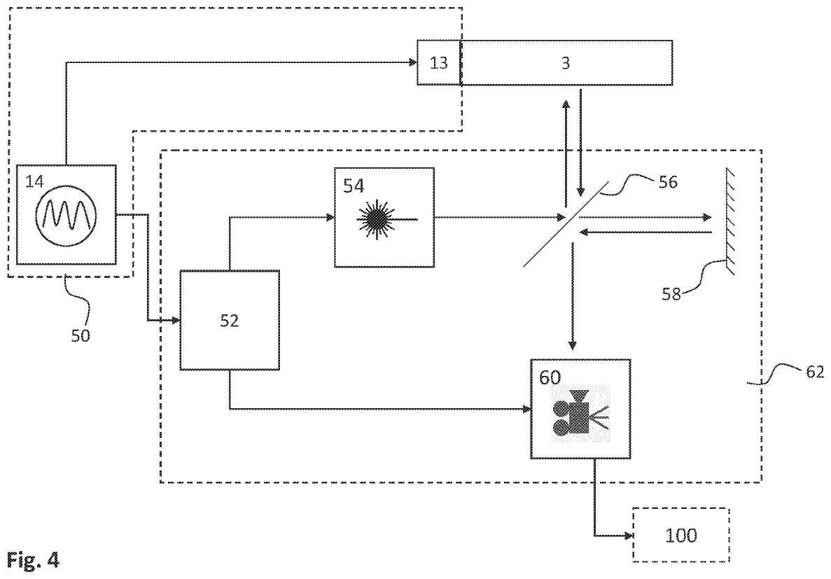

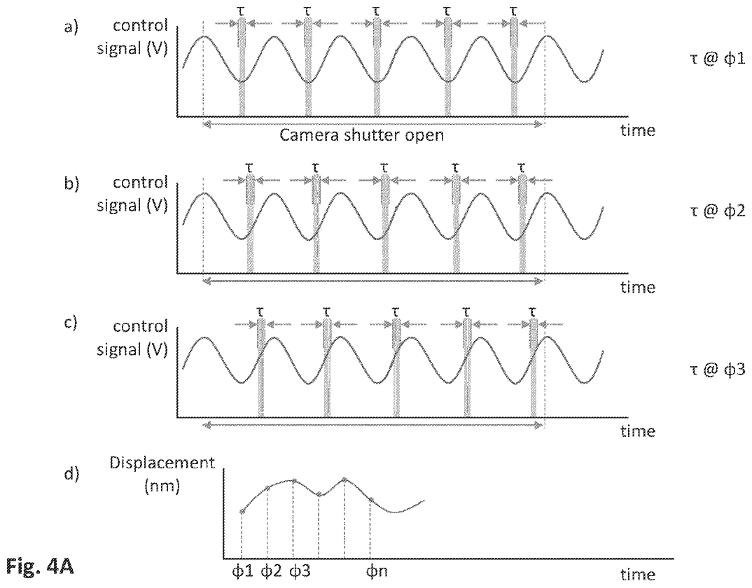

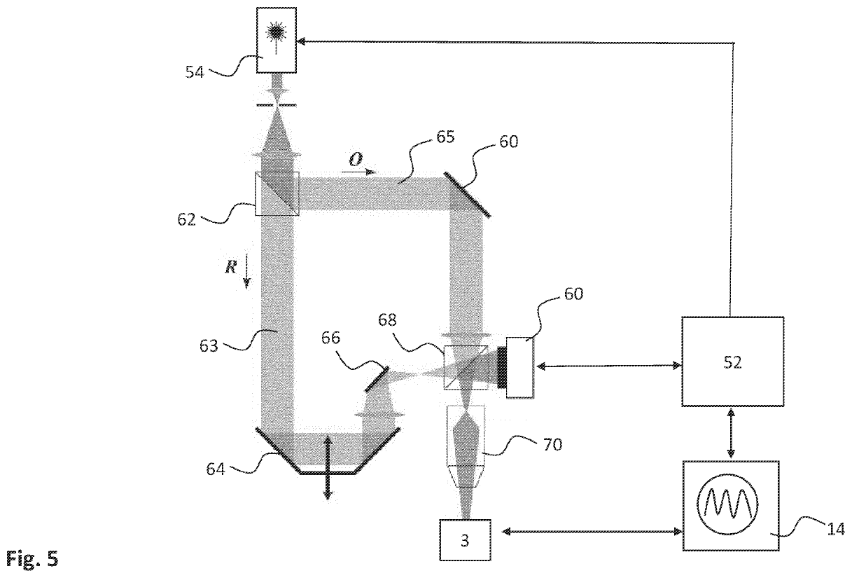

Preferably, the system for characterizing an acoustic-based particle manipulation device comprises an imaging system that is configured to measure the vibrations of the at least part of the surface using an imaging technique. In an embodiment, digital holographic microscopy is used. FIGS. 4 and 5 schematically illustrate such embodiments.

FIG. 4 schematically illustrates a system for characterizing an acoustic-based particle manipulation device 2 as described herein. The system comprises a signal provisioning system 50, a vibration measurement system 62, and, optionally, a data processing system 100. The signal provisioning system 50 is configured to provide a driving signal to the sample holder 3 for generating an acoustic field in the fluid-medium-containing holding space 5 that is suitable for driving a particle that sits at the wall surface portion 17, away from the wall surface portion 17. The vibration measurement system 62 is configured to measure vibrations of at least part of a surface of the sample holder caused by said driving signal. Preferably, vibrations of the wall surface portion 17 are measured. Preferably this is the glass/fluid medium interface on the inside of the channel. Before a typical experiment, this surface is functionalized using target cells. In this case, still, the characterization may be performed based on measurements of vibrations of the wall surface portion 17 without having any functionalized layer on it. This turns out to still be predictive of chip performance. The method could in principle also be applied after a surface functionalization. In this case, during the characterization vibrations of the wall surface portion 17 may be measured and/or vibrations of the surface formed by functionalized layer 10 as long as the DHM can focus on the right surface. It could be that chips are pre-functionalized in the factory e.g. with a poly-l-lysine coating to improve later sticking of cells. Such a functionalization would still allow to measure vibrations of the wall surface portion 17.

The optional data processing system 100 is configured to determine, based on the measured vibrations of said at least part of the surface, for each position out of a plurality of positions at the wall surface portion and in the holding space, a value for the acoustic force that is caused by said driving signal and that acts in a direction away from the wall surface portion, preferably the direction being substantially orthogonal to the wall surface portion.

In the depicted embodiment, the signal provisioning system 50 comprises a system 14 that comprises a power source and a control signal generator. System 14 outputs a control signal, such as a varying voltage signals, to an acoustic wave generator 13. System 14 may also provide a synchronization signal to either the camera 60 or the light source 54 in order to allow synchronization of the imaging with the acoustic wave (e.g. by stroboscopic illumination or by gating the camera exposure in synchrony with the acoustic wave) in order to sample multiple images at different phases of the periodic acoustic wave such that the periodic surface displacements can be accurately reconstructed. Multiple short pulses of minimal 7.5 ns for example may be used and measure those pulses at specific phase offsets and integrate them over one camera image to get enough signal on the camera. FIG. 4A illustrates such synchronization. Figures a, b and c show that multiple short pulses of length T are measured during the time that the camera shutter is open (indicated by the double arrow), wherein each laser pulse is at the same phase of the periodic control signal controlling an acoustic wave generator. Figures b and c show this for different phase offsets. In this manner, enough signal can be obtained for each phase offset and the vibration of the surface can be reconstructed as shown in figure d.

The acoustic wave generator 13 may be a piezo element that is attached to the sample holder 3. The acoustic wave generator may be any transducer that is configured to transduce control signals, such as electrical control signals, into mechanical vibrations. These mechanical vibrations can then vibrate the sample holder 3 such that an acoustic wave, e.g. a standing acoustic wave, is generated in the sample holder, in particular in the fluid medium that sits in the holding space.

Digital holographic microscopy may for example be implemented by providing a (coherent) light source 54 and a beam splitter 56 which splits the light from the light source into a sample arm sending the light to the sample 3 and a reference arm sending the light to a mirror 58. The light reflected back from the sample 3 is recombined with the light reflected back by the mirror 58 in the reference arm using the beam splitter 56 and is sent to camera 60 where the light fields interfere to form an interferogram or hologram. By e.g. introducing an angle on mirror 58 an angle offset between the reflected light fields can be introduced which sets up a fringe pattern (alternating lines of high and low intensity) in the off-axis hologram. Any surface deformations (either static or dynamically changing) can then be picked up as modulations of the fringe pattern (e.g. as deformations in the carrier fringe pattern). Reconstruction of the phase of the reflected sample wave (which allows reconstruction of the surface topography) can be performed either by obtaining multiple holograms at various phase delays between sample and reference arms (e.g. by stepping the mirror 58) or by multiplying the hologram with a computed replica of the reference wave as described by {Cuche et al. Applied Optics/Vol. 38, No. 34/1 Dec. 1999}.

The sample arm can contain a microscope objective in order to provide a (magnified) image of the surface deformations. Synchronizing either the camera exposure or the light source illumination with a periodic deformation of the sample surface of interest and varying the phase between the driving signal and the synchronization trigger allows the acquisition of off-axis holograms at different phases of the periodic deformation as explained with reference to FIG. 4A. This enables a full reconstruction of the periodic deformation.

Holography imaging techniques are well known in the art. Also see for example the following references {E. Archbold and A. E. Ennos, Nature 217 (1968) 942} and {B. M. Watrasiewicz and P. Spicer, Nature 217 (1968) 1142} and {P. Shajenko and C. D. Johnson, Appl. Phys. Lett. 13 (1968) 44} and {Hariharan et Oreb, Optics Communications, Volume 59, number 2, Aug. 15, 1986} and {Cuche et al. Applied Optics/Vol. 38, No. 34/Dec. 1, 1999}.

FIG. 5 schematically depicts an alternative setup for off-axis digital holographic microscopy using a Mach-Zehnder interferometer instead of the Michelson interferometer of FIG. 4. A light source 54 creates a beam of light (e.g. laser light) which is sent through a beam expander with an optional spatial filter to create a clean TEM00 mode. A beam splitter 62 splits the light into a reference arm Rand a sample or object arm O. The object arm light 65 is deflected by mirror 60, focused by a lens and transmitted through a second beam splitter 68 onto the back focal plane of an objective 70 which illuminates the sample 3. The light back reflected from the surface of interest on which the objective is focused is collected by the same objective 70 and reflected by beam splitter 68 onto a digital camera 60. The reference arm light beam 63 is directed by retroreflector 64 onto a lens and a mirror 66 through the beam splitter 68 onto the digital camera where the combination of the reference arm light and the light reflected from the sample forms a digital hologram. By introducing an angle between the light reflected from the sample and the reference arm light (e.g. by tilting mirror 66 away slightly from 45 degrees) an off-axis hologram can be formed which allows reconstruction of the reflected wavefront and the sample topography according to Cuche 1999. A controller 52 (e.g. a PC) may be used to control a waveform generator 14 that drives a transducer on device 3 in order to create a resonant standing wave in the holding space of the device. The controller may also control the digital camera 60 and the light source 54 and may perform the surface vibration reconstruction based on the recorded off-axis holograms.

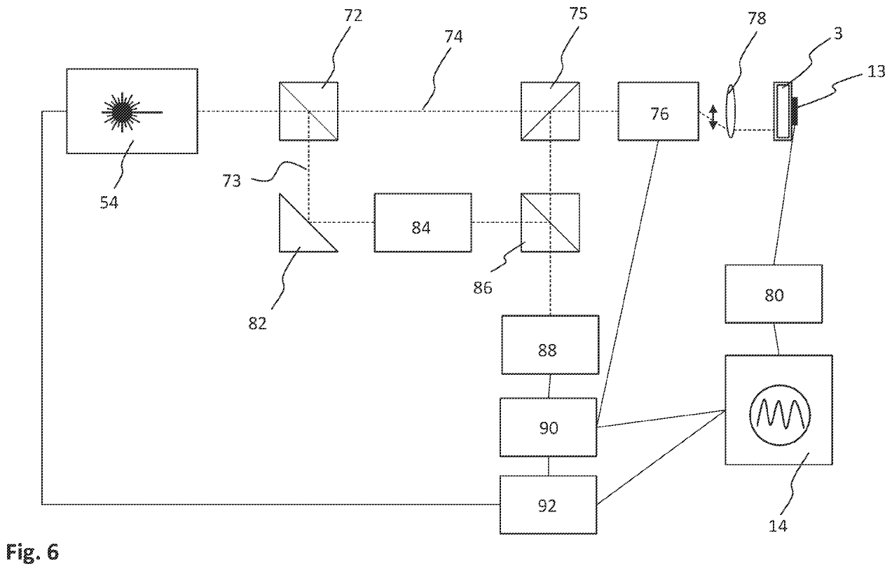

In an embodiment, measuring the vibrations of the at least part of the surface is performed using laser doppler vibrometry. FIG. 6 shows a system according to an embodiment, which system comprises a laser doppler vibrometer.

A light beam is generated by a laser source 54, such as a helium-neon laser, laser diode, fiber laser, and/or Nd: YAG laser, and is split into a reference arm 73 and a sample arm 74 by beam splitter 72. The sample arm beam 74 is send through an XY scanner 76 and an objective lens 78 and is focussed onto the target surface of the sample holder 3 of which vibrations are going to be measured. Beam splitter 75 is used to direct the sample light backreflected from the sample holder towards the detector 88. Combiner 86 combines the sample and reference beams. By introducing a frequency shifter 84, e.g. a Bragg cell or acousto-optic modulator (AOM), a known frequency shift can be applied to the reference arm 73, such as a shift of 30-40 MHZ, and a heterodyne carrier frequency can be set up which facilitates distinguishing positive from negative displacements and/or velocities of the surface under investigation. The detector 88 may be coupled to a Data acquisition subsystem 90 (DAQ system) which may e.g. contain an analog-to-digital converter (ADC). The DAQ system 90 may also contain e.g. a preamplifier and/or filter system (not shown). A controller 92 may control the DAQ system and may be used to store, analyze and visualize the detector signals. The controller 92 may also control the XY scanner 76 e.g. through the DAQ system 90. The controller 92 and DAQ systems 90 may further be connected to e.g. a function generator 14 which sends a signal to the transducer 13 (e.g. through a suitable amplifier 80) to control the vibrations of the sample holder. The controller 92 and DAQ system 90 may be used to synchronize the vibration to the data acquisition and the scanning. The controller 92 may also control the laser source 54.

The motion of the surface adds a Doppler shift to the sample beam. Light scatters from the target in all directions, but some portion of the light is ultimately collected by the detector 88, that can respond to the beat frequency between the two beams.

The output of the detector 88 is for example a standard frequency modulated (FM) signal, with the Bragg cell frequency as the carrier frequency, and the Doppler shift as the modulation frequency. This signal can be demodulated to derive the velocity vs. time of the vibrating surface.

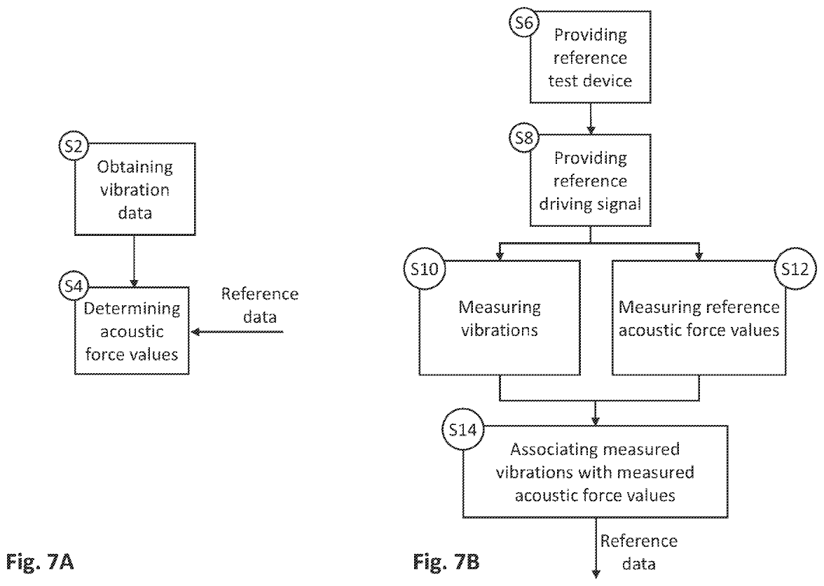

One aspect of this disclosure relates to a computer-implemented for characterizing an acoustic-based particle manipulation device. FIG. 7A illustrates such method according to an embodiment. Herein, step S2 comprises obtaining vibration data indicating, for each point of a plurality of points on a surface of the sample holder, a measured amplitude value indicative of amplitude with which the point in question vibrates as a result of a driving signal provided to the sample holder. Such vibration data may be output by a vibration measurement system as described herein. Due to manufacturing variations, which areas of for example the wall surface portion vibrate heavily (with large amplitudes) and which areas only slightly (with small amplitudes) differs from particle manipulation device to particle manipulation device. Hence, the vibration data differs from particle manipulation device to particle manipulation device.

Then, step S4 comprises, based on the vibration data, determining, for each position out of a plurality of positions at a wall surface portion of a holding space, the plurality of positions being in the holding space, a value for the acoustic force that is caused by said driving signal and that acts in a direction away from the wall surface portion, preferably the direction being substantially orthogonal to the wall surface portion. In this step, preferably, use is made of reference data associating reference amplitude values with reference values for the acoustic force. As such, the reference data may be understood to link the obtained vibration data to actual values for the derivative of acoustic pressure distribution along a direction away from the wall surface portion.

Of course, the method for characterizing the acoustic-based particle manipulation device can be performed for different driving signals, that differ for example in frequency and/or power. This allows to determine, for a given position in the holding space of the calibrated particle manipulation device, for a given driving signal, the value of the acoustic force in a direction away from the wall surface portion. To illustrate, during a characterization, the frequency of the driving signal may be increased in steps of 200 Hz, e.g. in a range 5 KHz around a resonance frequency of the device. Then, for each frequency, an acoustic force map may be obtained. The method may also comprise determining an average acoustic force map based on these several determined acoustic force maps. such average acoustic force maps indicates for each position, an average value of acoustic force, which average value an average of the values that are indicated for the position in question by the several determined acoustic force maps. In principle, multiple resonance or off resonance frequencies could be measured to fully calibrate the chip for any driving frequency.

FIG. 7B illustrates how such reference data may be obtained by performing measurements on a reference acoustic-based particle manipulation device. Such reference particle manipulation device is preferably similar, e.g. in terms of the dimensions of the sample holder, holding space, configuration, acoustic wave generator, fluid medium, et cetera, to the acoustic-based particle manipulation device that is to be characterized based on the reference data. Thus, such reference particle manipulation device comprises a reference holding space containing a reference fluid medium, the reference holding space comprising a reference wall surface portion and herein, preferably, the reference holding space is similar to the holding space of the acoustic-based particle manipulation device that is to be characterized using the reference data, the reference fluid medium is preferably the same as the fluid medium in the holding space of the to-be-characterized particle manipulation device, the fluid temperature for the reference measurement is preferably the same as the fluid temperature in the holding space of the to-be-characterized particle manipulation device, the reference wall surface is preferably the same as the wall surface of the to-be-characterized particle manipulation device. Step S6 indicates the step of providing such reference acoustic-based particle manipulation device.

Step S8 comprises providing a reference driving signal to the reference sample holder for generating a reference acoustic field in the reference holding space that is suitable for driving a particle that sits at the reference wall surface portion, away from the reference wall surface portion. Preferably, the reference driving signal is the same as the driving signal that will be applied to the to-be-characterized particle manipulation device during the characterization and for which driving signal the values for the acoustic force are determined.

Then, while the reference driving signal is applied, the vibrations of a reference surface of the reference sample holder caused by the reference driving signal are measured. In parallel, step S12 is performed which comprises, measuring, for each position out of a plurality of positions at the reference wall surface portion and in the reference holding space, a reference value for the acoustic force, caused by said reference driving signal acting in a direction away from the reference wall surface portion, preferably the direction being substantially orthogonal to the wall surface portion. Step S12 may be performed by performing bead tracking experiments as for example described in the Background section of this disclosure.

The vibration data resulting from step S10 and the values of the acoustic force of step S12 may be associated with each other in a step S14 in order to obtain the reference data. The reference data may then in particular associate amplitude values with respective values of acoustic force in a direction away from the sample surface. Such amplitude value—acoustic force value pair in the reference data does not need be associated with for example an xy-position of the wall surface portion.

The bead tracking experiments that may be performed in step S12 determine the acoustic pressure distribution at positions slightly above the wall surface portion 17 because the starting position of the tracked particles is slightly above the wall surface portion 17 due to these particles sitting on a functionalized layer on the wall surface portion 17 (also see FIG. 2A). In such case, the reference data thus link amplitude values to respective values of the acoustic force—in a direction away from the wall surface portion 17—for positions present slightly above the wall surface portion 17.

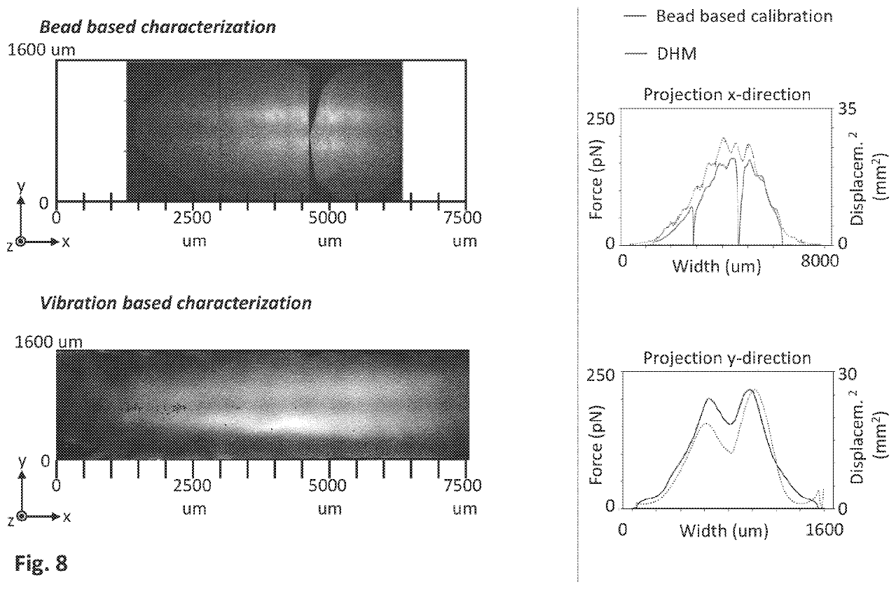

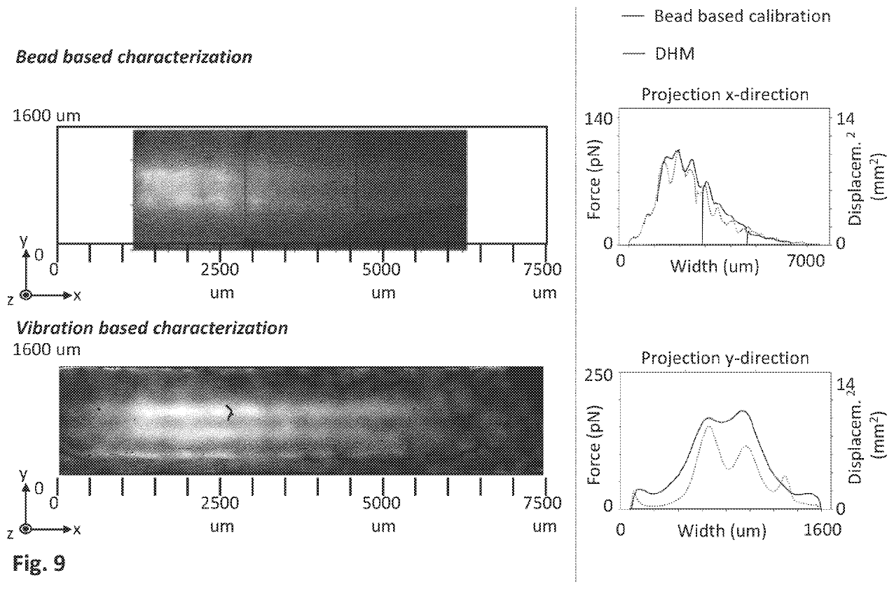

FIGS. 8 and 9 illustrate that the method for characterizing acoustic-based particle manipulation device as disclosed herein yields results that are similar to results obtained when bead based characterization is performed.

The top left heat map illustrates a characterization of a particle manipulation device that was obtained by performing bead tracking experiments. The heat map shows for each of a plurality of positions at a wall surface portion of the particle manipulation device, a value of the acoustic force in a direction away from the wall surface portion, e.g. in a direction parallel to the z-direction as indicated. In this heat map, brighter regions are associated with higher acoustic force values. Note that the heat map is formed by three separate sub-heat maps. This is due to the fact that three separate bead tracking experiments needed to be performed to characterize substantially the entire wall surface portion.

The bottom left heat map illustrates a characterization of the same particle manipulation device. However, here the characterization was obtained using a method disclosed herein for characterizing a particle manipulation device, thus a method that involves measuring vibrations of a surface of the sample holder. In this case, vibrations of the wall surface portion were measured. Brighter regions are associated with larger amplitude values (in the z-direction) with which the wall surface portion vibrates.

Based on these heat maps alone, it can already be seen that the regions that have high amplitude values also have high acoustic force values for the acoustic pressure in a direction away from the wall surface portion 17. Due to this correlation, the bottom left heat map thus indicates for each of a plurality of positions at the wall surface portion, a value for the acoustic force in a direction away from the wall surface portion.

To investigate the similarity between the two heat maps further, the graphs on the right hand side show projections of the average force (pN, axis on the left) and average displacement squared (nm², axis on the right) onto the x-axis (top graph) and y-axis (bottom graph). The indicated force is the force that a a silica bead with a ca 10.1 micrometer diameter (standard deviation 0.1 micrometer) would experience siting in a fluid consisting of 1× PBS (Phosphate-buffered saline) solution supplemented with 0.02% w/v Pluronic f127+0.02% w/v Casein solution at a sample temperature in a range of 25-37 degrees Celsius. Thus, the top graph indicates for each of x-position, x, in the range 0-7500 um, an average acoustic force in pN as determined based on particle tracking experiments and an average amplitude in nm as measured by a vibration measurement system, along a line segment from (x,0 um) to (x,1600 um). The bottom graph indicates for each of y-position, y, in the range 0-1600 um, an average acoustic force in pN and an average displacement in nm, along a line segment from (0 um,y) to (7500 um,y). These graphs show a clear correlation between the acoustic force and amplitude.

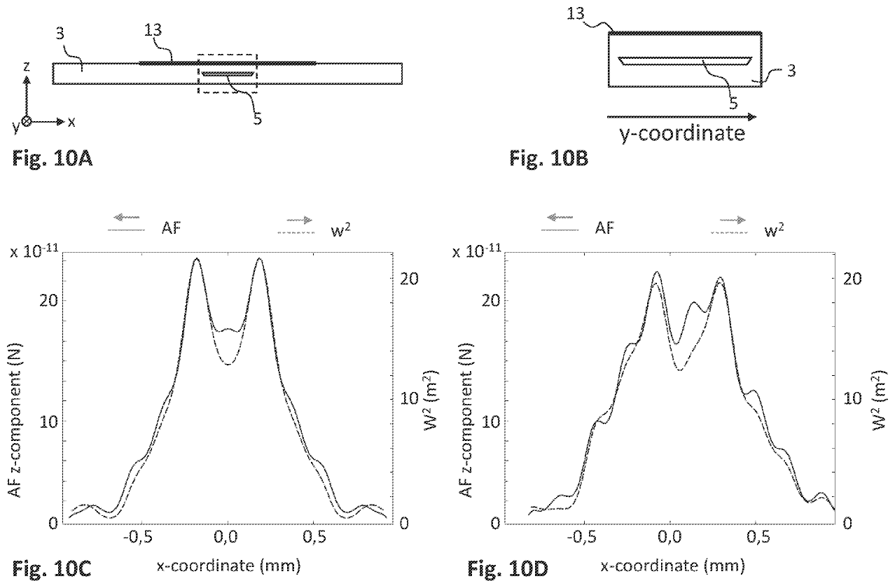

FIG. 10A shows the 2D model of a COMSOL model used to generate the FIGS. 10C and 10D. It shows a cross section of the device 2 which consists of a glass chip 3, also referred to herein as sample holder, with a thickness of 800 micrometers and a width of 14 mm. A channel 5, also referred to herein as a holding space 5, of 100 micrometers in height and 1980 micrometers in width, filled with water is located near the center of the chip 3. The piezo excitation, also referred to herein as the provisioning of the driving signal to the sample holder, is modeled as a piston mode (flat phase) vibration of the top surface of the glass 3 at the same location as the piezo 13 on the physical device. The size of the piezo 13 in the model is 7 mm. The excitation of the top surface has a frequency of 7.7425 MHz and a maximum velocity of 0.5 mm/s.

FIG. 10B shows a zoom of the dashed box in FIG. 10A. In the performed simulations, the displacement of the bottom surface of the channel 5 in the vertical direction is determined as a function of the location. The results are shown in FIGS. 10C and 10D.

Graphs 10C and 10D show the simulated Acoustic Force (AF) in the z-direction (solid line, axis on the left) and the squared displacement of the channel bottom (dashed line, axis on the right). The AF is calculated 5 micrometer above the channel bottom from the 2D simulated pressure field using the Gorkov Approximation on a polystyrene particle (r=5 um, density=1050 kg/m³, speed of sound=2500 m/s). The displacement of the bottom of channel 5, w, is the component of displacement perpendicular to the channel bottom, i.e. the displacement in the z-direction, as a function of x-position as indicated, and this simulates the vibrations of the bottom surface of channel 5.

The AF-simulation (solid line) and vibration simulation (dashed line) in FIG. 10C were obtained with the geometry as presented in FIGS. 10A and 10B (with the excitation over the 7 mm field under the simulated piezo centered over the channel 5. What is clear is that the AF correlates strongly with the square of the bottom surface displacement. The simulations in FIG. 10B were obtained by simulating a shift of the piezo by 0.1 mm to the right. Such a misalignment of the piezo causes changes to the details of the cross section of the force profile across the channel 5, but, clearly, the proportionality between the AF and the squared displacement is conserved.

Determining values of acoustic force based on vibrations of a surface of the sample holder may thus be performed, in particular, based on the squared amplitudes of such vibrations of respective points on the surface.

It should be appreciated that the sample holder of FIG. 2A is not to scale. Typically, the distance d3 between surface 17 and surface 18 is much larger than the distance d1 between surface 24 and surface 20. Further, distance d2 between the top surface 20 and bottom surface 17 of the holding space is much smaller than distance d3. Typical values for d1, d2 and d3 are approximately 400 microns, approximately 95 microns and approximately 300 microns, respectively.

As indicated above, the surface of which the vibrations are measured, is relatively close to the holding space 5 so that the acoustic pressure in the holding space 5 can be accurately determined. Therefore, in a preferred embodiment, the surface of which the vibrations are measured is the wall surface portion 17 and/or the surface formed by a functionalized layer 10 on the wall surface portion 17. However, since these surfaces are relatively close to surface 20, it may be difficult to obtain high quality measurements of these surfaces. This may especially be the case if an imaging and/or optical technique is used to measure the vibrations. Then, any light that is used in such techniques and that reflects back from the surface of interest, i.e. the surface of which the vibrations are to be measured, may interfere with light that reflects back (unintentionally) from other surfaces near the surface of interest. If the surface of interest is the wall surface portion 17 and/or the surface formed by a functionalized layer 10 on the wall surface portion 17, then surface 20 may cause such interference, since it is relatively close by. This interference may cause artifacts in the measurement results.

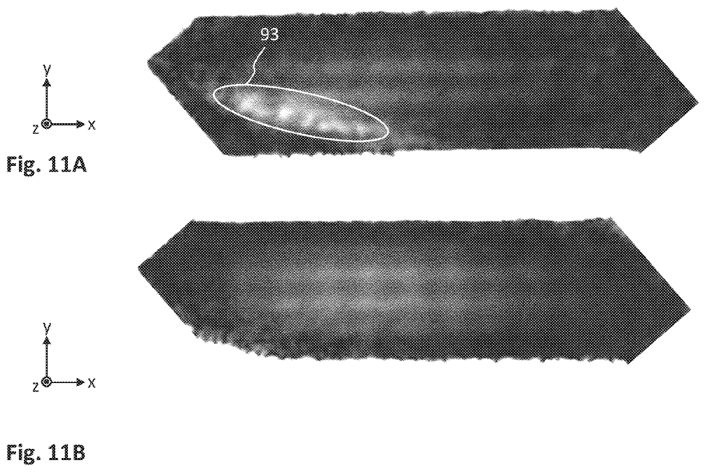

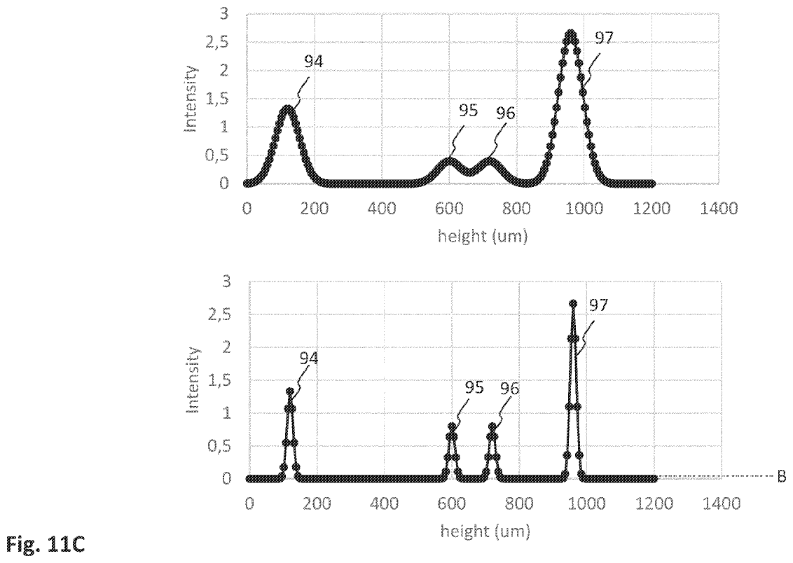

FIG. 11A is an example of a heat map that contains such artifact at region 93. As a side note, these heat maps indicate for each position a measured amplitude of the vibration, wherein the measured amplitudes range from 0 nm (black) to 7 nm (white). The measured surface in question did not have large vibrations at region 93, although the heat map shows a high intensity region here. This artifact is caused by interference between light reflected back from the wall surface portion and light reflected back from the top surface 20 of the holding space 5 17 (see FIG. 2A). The coherence length of the light that was used to measure the vibrations of the wall surface portion 17 was 400 micrometer.

In contrast, everything else being equal, FIG. 11B shows the heat map that was obtained when a coherence length of (only) 15 micrometers was used. FIG. 11B does not contain any artifact. Hence, a shorter coherence length of light that is used to measure the vibrations, yields more accurate results. This holds irrespective of whether digital holographic microscopy is used to measure the vibrations, or laser doppler vibrometry, or phase sensitive optical coherence tomography, or optical coherence phase microscopy, or any other optical technique.

Using a relatively small coherence length in principle reduces the field of view. A small tilt of the sample with respect to the incoming light may cause the sample to be out of focus. To alleviate this problem, the system may comprise a movable stage, such as a tilt stage and/or a tip-tilt stage known in the art, that is configured to adapt the orientation of the surface with respect to incoming light. This allows to accurately position the surface and/or the acoustic-based particle manipulation device with respect to the incoming light so that the surface of interest is properly in focus.

In order to illustrate that a shorter coherence length can yield more accurate results, FIG. 11C shows a comparison between two measurements. In each measurement, using digital holographic microscopy, a scan was made in the z-direction (see e.g. FIG. 2A). Each scan involved focusing the light of the DHM at different z-positions (indicated on the horizontal axis) and measuring the amount of signal that was received back. During the scan, no vibrations were applied to the sample holder (static conditions). It should be appreciated that instead of changing the focus of the incoming light, also the length of the reference arm that is present in the setup in question (e.g. described above) may be adapted. In any case, everything else being equal, the light that was used for the first measurement (top graph) had a coherence length of 400-500 micrometers, while the light that was used for the second measurement (bottom graph) had a coherence length of approximately 15 micrometers.

The measurements show the same peaks, namely peak 94 associated with the light being focused on surface 24 (see FIG. 2A), peak 95 associated with the light being focused on surface 20, peak 96 associated with the light being focused on surface wall surface portion 17, and peak 97 associated with the light being focused on surface 18. However, due to the smaller coherence length, the peaks of the second measurement (bottom) are narrower. As a result, peaks 95 and 96 no longer overlap, which may be understood as that the value of the signal between the peaks reaches a baseline value indicated by B. As referred to herein, a baseline value may be understood as the value of the signal that is received back when the light does not reflect back from a surface. In any case the non-overlapping peaks indicate that, if the coherence lengths 15 micrometers is used, substantially no interference occurs between light reflected back from the wall surface portion 17 and light reflected back from the top surface 20 of the holding space 5. The above-described measurements can thus be used to select an appropriate value for the coherence length for any given particle manipulation device and/or for any given sample holder.

In general, the coherence length of the light that is used to measure vibrations of the surface of interest is preferably selected such that substantially no interference occurs between light reflected back from the surface of interest, i.e. the surface of which vibrations are to be measured, and light reflected back from other surfaces of the particle manipulation device, e.g. of the sample holder. Additionally or alternatively, the coherence length is preferably selected such that, if a scan is performed in the z-direction as described above, the peak associated with the surface of interest does not overlap with another peak of another surface.

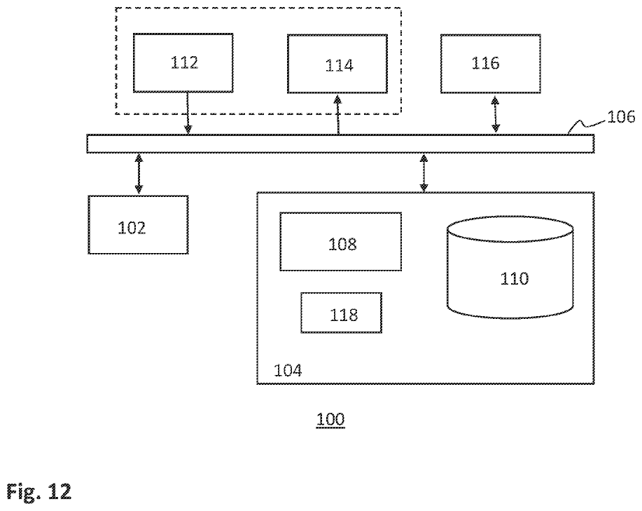

FIG. 12 depicts a block diagram illustrating a data processing system according to an embodiment.

As shown in FIG. 12, the data processing system 100 may include at least one processor 102 coupled to memory elements 104 through a system bus 106. As such, the data processing system may store program code within memory elements 104. Further, the processor 102 may execute the program code accessed from the memory elements 104 via a system bus 106. In one aspect, the data processing system may be implemented as a computer that is suitable for storing and/or executing program code. It should be appreciated, however, that the data processing system 100 may be implemented in the form of any system including a processor and a memory that is capable of performing the functions described within this specification.

The memory elements 104 may include one or more physical memory devices such as, for example, local memory 108 and one or more bulk storage devices 110. The local memory may refer to random access memory or other non-persistent memory device(s) generally used during actual execution of the program code. A bulk storage device may be implemented as a hard drive or other persistent data storage device. The processing system 100 may also include one or more cache memories (not shown) that provide temporary storage of at least some program code in order to reduce the number of times program code must be retrieved from the bulk storage device 110 during execution.

Input/output (I/O) devices depicted as an input device 112 and an output device 114 optionally can be coupled to the data processing system. Examples of input devices may include, but are not limited to, a keyboard, a pointing device such as a mouse, a touch-sensitive display, or the like. Examples of output devices may include, but are not limited to, a monitor or a display, speakers, or the like. Input and/or output devices may be coupled to the data processing system either directly or through intervening I/O controllers.

In an embodiment, the input and the output devices may be implemented as a combined input/output device (illustrated in FIG. 12 with a dashed line surrounding the input device 112 and the output device 114). An example of such a combined device is a touch sensitive display, also sometimes referred to as a “touch screen display” or simply “touch screen”. In such an embodiment, input to the device may be provided by a movement of a physical object, such as e.g. a stylus or a finger of a user, on or near the touch screen display.

A network adapter 116 may also be coupled to the data processing system to enable it to become coupled to other systems, computer systems, remote network devices, and/or remote storage devices through intervening private or public networks. The network adapter may comprise a data receiver for receiving data that is transmitted by said systems, devices and/or networks to the data processing system 100, and a data transmitter for transmitting data from the data processing system 100 to said systems, devices and/or networks. Modems, cable modems, and Ethernet cards are examples of different types of network adapter that may be used with the data processing system 100.

As pictured in FIG. 12, the memory elements 104 may store an application 118. In various embodiments, the application 118 may be stored in the local memory 108, the one or more bulk storage devices 110, or apart from the local memory and the bulk storage devices. It should be appreciated that the data processing system 100 may further execute an operating system (not shown in FIG. 12) that can facilitate execution of the application 118. The application 118, being implemented in the form of executable program code, can be executed by the data processing system 100, e.g., by the processor 102. Responsive to executing the application, the data processing system 100 may be configured to perform one or more operations or method steps described herein.

In one aspect of the present invention, the data processing system 100 may represent a function generator or any controller described herein.

Various embodiments of the invention may be implemented as a program product for use with a computer system, where the program(s) of the program product define functions of the embodiments (including the methods described herein). In one embodiment, the program(s) can be contained on a variety of non-transitory computer-readable storage media, where, as used herein, the expression “non-transitory computer readable storage media” comprises all computer-readable media, with the sole exception being a transitory, propagating signal. In another embodiment, the program(s) can be contained on a variety of transitory computer-readable storage media. Illustrative computer-readable storage media include, but are not limited to: (i) non-writable storage media (e.g., read-only memory devices within a computer such as CD-ROM disks readable by a CD-ROM drive, ROM chips or any type of solid-state non-volatile semiconductor memory) on which information is permanently stored; and (ii) writable storage media (e.g., flash memory, floppy disks within a diskette drive or hard-disk drive or any type of solid-state random-access semiconductor memory) on which alterable information is stored. The computer program may be run on the processor 102 described herein.

The terminology used herein is for the purpose of describing particular embodiments only and is not intended to be limiting of the invention. As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of embodiments of the present invention has been presented for purposes of illustration, but is not intended to be exhaustive or limited to the implementations in the form disclosed. Many modifications and variations will be apparent to those of ordinary skill in the art without departing from the scope and spirit of the present invention. The embodiments were chosen and described in order to best explain the principles and some practical applications of the present invention, and to enable others of ordinary skill in the art to understand the present invention for various embodiments with various modifications as are suited to the particular use contemplated.

1. A method for characterizing an acoustic-based particle manipulation device, wherein
the acoustic-based particle manipulation device comprises a sample holder comprising a holding space containing a fluid medium, the holding space comprising a wall surface portion, the method comprising
providing a driving signal to the sample holder for generating an acoustic wave in the fluid-medium-containing holding space that is suitable for driving away a particle that sits at the wall surface portion away from the wall surface portion, and
measuring vibrations of at least part of a surface of the sample holder caused by said driving signal, and
based on the measured vibrations of said at least part of the surface, determining, for each position out of a plurality of positions at the wall surface portion and in the holding space, a value of an acoustic force that is caused by said driving signal and that acts in a direction away from the wall surface portion, preferably the direction being substantially orthogonal to the wall surface portion.

2. The method according to claim 1, wherein measuring the vibrations of the at least part of the surface is performed using an imaging technique.

3. The method according to claim 2, wherein measuring the vibrations of the at least part of the surface is performed using digital holographic microscopy.

4. The method according to claim 1, wherein measuring the vibrations of the at least part of the surface is performed using laser doppler vibrometry and/or phase sensitive optical coherence tomography and/or optical coherence phase microscopy.

5. The method according to claim 1, wherein each position out of the plurality of positions is at a distance from the wall surface portion.

6. The method according to claim 1, wherein measuring vibrations of the at least part of the surface comprises determining, for each point of a plurality of points on the surface, an amplitude value indicative of amplitude with which the point in question vibrates.

7. The method according to claim 1, wherein said surface is a surface in the holding space and/or is a surface at least partially defining the holding space and/or is a surface adjacent the holding space and/or is the wall surface portion.

8. The method according to claim 1, wherein the step of determining, for each position, a value of said acoustic force is performed based on reference data associating reference amplitude values with reference values for the acoustic force.

9. The method according to claim 8, further comprising
obtaining said reference data, this step comprising
providing a reference acoustic-based particle manipulation device comprising a reference sample holder comprising a reference holding space containing a reference fluid medium, the reference holding space comprising a reference wall surface portion, and
providing a reference driving signal to the reference sample holder for generating a reference acoustic wave in the reference holding space that is suitable for driving a particle that sits at the reference wall surface portion, away from the reference wall surface portion, and
measuring vibrations of at least part of a reference surface of the reference sample holder caused by said reference driving signal, and
measuring, for each position out of a plurality of positions at the reference wall surface portion and in the reference holding space, a reference value of an acoustic force that is caused by said reference driving signal and that acts in a direction away from the reference wall surface portion.

10. The method according to claim 1, further comprising
storing the determined values for the acoustic force on a non transitory computer-readable storage medium in association with respective indications of their respective associated positions in the holding space.

11. The method according to claim 1, comprising
providing a first driving signal to the sample holder for generating a first acoustic wave in the fluid-medium-containing holding space that is suitable for driving a particle that sits at the wall surface portion, away from the wall surface portion, and
measuring first vibrations of at least part of a surface of the sample holder caused by said first driving signal, and
based on the measured first vibrations of said at least part of the surface, determining, for each position out of a plurality of positions at the wall surface portion and in the holding space, a first value of the acoustic force that is caused by said first driving signal and that acts in a direction away from the wall surface portion, and
providing a second driving signal to the sample holder for generating a second acoustic wave in the fluid-medium-containing holding space that is suitable for driving a particle that sits at the wall surface portion, away from the wall surface portion, and
measuring second vibrations of at least part of a surface of the sample holder caused by said second driving signal, and
based on the measured second vibrations of said at least part of the surface, determining, for each position out of a plurality of positions at the wall surface portion and in the holding space, a second value for the acoustic force that is caused by said second driving signal and that acts in a direction away from the wall surface portion.

12. A system comprising
an acoustic-based particle manipulation device comprising a sample holder comprising a holding space containing a fluid medium, the holding space comprising a wall surface portion, and
a signal provisioning system for providing a driving signal to the sample holder for generating an acoustic field in the fluid-medium-containing holding space that is suitable for driving a particle that sits at the wall surface portion, away from the wall surface portion, and
a vibration measurement system for measuring vibrations of at least part of a surface of the sample holder caused by said driving signal.

13. A computer-implemented method for characterizing an acoustic-based particle manipulation device, the device comprising a sample holder comprising a holding space containing a fluid medium, the holding space comprising a wall surface portion, the method comprising
obtaining vibration data indicating, for each point of a plurality of points on a surface of the sample holder, a measured amplitude value indicative of amplitude with which the point in question vibrated as a result of a driving signal provided to the sample holder, and
based on the vibration data, determining, for each position out of a plurality of positions at a wall surface portion of a holding space, the plurality of positions being in the holding space, a value of the acoustic force that is caused by said driving signal and that acts in a direction away from the wall surface portion.

14. A data processing system that is configured to perform the method according to claim 13.

15. A computer program comprising instructions, which, when the instructions are executed by a data processing system, cause the data processing system to perform the method according to claim 13.

16. A non-transitory, computer-readable storage medium obtainable by performing the method according to claim 10.

17. The computer-implemented method of claim 13, wherein direction of the acoustic force is substantially orthogonal to the wall surface portion.

18. The method of claim 11, wherein direction of the acoustic force is substantially orthogonal to the wall surface portion.

19. The method of claim 9, wherein direction of the acoustic force is substantially orthogonal to the wall surface portion.

20. The system of claim 12 and further comprising:
a data processing system configured to determine, based on the measured vibrations of said at least part of the surface, for each position out of a plurality of positions at the wall surface portion and in the holding space, a value for the acoustic force that is caused by said driving signal and that acts in a direction away from the wall surface portion, preferably the direction being substantially orthogonal to the wall surface portion.
